(19) United States
(12) Patent Application Publication
(10) Pub. No.: US 2015/0149092 A1
(43) Pub. Date: May 28, 2015

(54) WEARABLE INTERFACE FOR DRILLING INFORMATION SYSTEM

(71) Applicant: National Oilwell Varco, L.P., Houston, TX (US)

(72) Inventors: Blake HANCHER, Columbus, TX (US); Robert Eugene MEBANE, Austin, TX (US)

(73) Assignee: National Oilwell Varco, L.P., Houston, TX (US)

(21) Appl. No.: 14/089,419

(22) Filed: Nov. 25, 2013

(51) Int. Cl. E21B 49/00; A61F 9/02; A61F 11/06; G01V 99/00

(52) U.S. Cl. CPC E21B 49/003; G01V 99/00; A61F 9/02; A61F 11/06
USPC 702/9

(57) ABSTRACT

A drilling information system comprises a drilling component having an operating status and a drilling control module communicatively coupled to the drilling component and operable to store data indicating the operating status of the drilling component. A wearable interface is communicatively coupled to the drilling control module and is operable to selectively indicate the operating status of the drilling component.

### CROSS-REFERENCE TO RELATED APPLICATIONS

None

### BACKGROUND

This disclosure relates generally to methods and apparatus for distributing information from a drilling information system. More specifically, this disclosure relates to methods and apparatus for distributing information regarding the operation of a drilling system to a user using a wearable interface.

To recover hydrocarbons from subterranean formations, wells are generally constructed by drilling into the formation using a rotating drill bit attached to a drill string. A fluid, commonly known as drilling mud, is circulated down through the drill string to lubricate the drill bit and carry cuttings out of the well as the fluid returns to the surface. The particular methods and equipment used to construct a particular well may vary extensively based on the environment and formation in which the well is being drilled. Many different types of equipment and systems are used in the construction of wells including, but not limited to, rotating equipment for rotating the drill bit, hoisting equipment for lifting the drill string, pipe handling systems for handling tubulars used in construction of the well, including the pipe that makes up the drill string, pressure control equipment for controlling wellbore pressure, mud pumps and mud cleaning equipment for handling the drilling mud, directional drilling systems, and various downhole tools.

Monitoring the varying machinery operating on a drilling rig is often accomplished through a variety of control systems. In many instances, each of the different control systems has selected data that is monitored by rig personnel. Distributing this data in an understandable and actionable way to rig personnel is becoming increasingly complex as the instrumentation of the drilling process increases.

In many systems, data is distributed via graphical interfaces that display selected operating parameters of critical equipment for observation by rig personnel. The graphical interface may include analog or digital gauges, visual alarm indicators, display screens, indicator lights, and other graphical interface. These graphical interfaces are generally located in control rooms, driller's cabins, or other areas separated from the working environment. Because these graphical interfaces are separated from the working environment, in many situations only certain rig personnel will be in visual range of the graphical interface and be able to access the information displayed thereof.

Another limitation of many current systems is a limitation on physical space for the display of information. Physical space, both within these separated locations and on the graphical interfaces themselves, is often restricted and can limit the data that can be displayed and/or the location in which it is displayed. Even when all needed data is displayed, the rig personnel monitoring the equipment are often forced to look away from the operating equipment in order to read any data on a graphical interface.

Thus, there is a continuing need in the art for methods and apparatus for distributing information from a drilling control system.

### BRIEF SUMMARY OF THE DISCLOSURE

A drilling information system comprises a drilling component having an operating status and a drilling control module communicatively coupled to the drilling component and operable to store data indicating the operating status of the drilling component. A wearable interface is communicatively coupled to the drilling control module and is operable to selectively indicate the operating status of the drilling component.

In certain embodiments, the wearable interface comprises a wearable frame including (a) a location sensor that generates location data indicating a location of the wearable frame on a drilling rig, (b) a field-of-view sensor that generates field-of-view data indicating a field-of-view of a user wearing the wearable frame, (c) a transmitter that transmits the location data and field-of-view data to the drilling control module, (d) a receiver to receive operating data from the drilling control module that indicates the operating status of the drilling component that is disposed within the field-of-view, and (e) a see-through display screen to display the operating data proximate to the drilling component. In some embodiments, the drilling information further comprises an RFID antenna coupled to the wearable frame and operable to detect an RFID tag mounted on the drilling component. In some embodiments, an RFID tag is coupled to the wearable frame and operable to identify the wearable interface. In some embodiments, a speaker is coupled to the wearable frame. In some embodiments, a microphone is coupled to the wearable frame. In some embodiments, the operating data that is displayed on the see-through display screen is dependent on a location of the wearable interface on the drilling rig. In some embodiments, the wearable interface provides hearing and eye protection to a user.

In certain embodiments, a wearable interface comprises a wearable frame, a location sensor that generates location data indicating a location of the wearable frame on a drilling rig, a field-of-view sensor that generates field-of-view data indicating a field-of-view of a user wearing the wearable frame, a transmitter to transmit the location data and field-of-view data to a drilling control module, a receiver to receive operating data from the drilling control module, wherein the operating data indicates an operational status of a drilling component that is disposed within the field-of-view, and a see-through display screen to display the operating data proximate to the drilling component.

In some embodiments, the wearable interface further comprises an RFID antenna coupled to the wearable frame, wherein the RFID antenna is operable to detect an RFID tag mounted on the drilling component. In some embodiments, the wearable interface further comprises an RFID tag coupled to the wearable frame and operable to identify the wearable interface. In some embodiments, a speaker is coupled to the wearable frame. In some embodiments, a microphone is coupled to the wearable frame. In some embodiments, the operating data displayed on the see-through display screen is dependent on a location of the wearable interface on the drilling rig. In some embodiments, the wearable interface provides hearing and eye protection to a user.

In certain embodiments, a method for displaying data comprises disposing a wearable frame on a user located on a drilling rig, generating location data indicating a location of the user on the drilling rig, generating field-of-view data indicating a field-of-view of the user, transmitting the location data and field-of-view data to a drilling control module, receiving operating data from the drilling control module, wherein the operating data indicates an operational status of a drilling component that is disposed within the field-of-view, and displaying the operating data on a see-through display screen coupled to the wearable frame.

In some embodiments, the method further comprises receiving data identifying the drilling component from an RFID tag mounted on the drilling component. In some embodiments, the method further comprises transmitting data identifying the wearable frame with an RFID tag mounted on the wearable frame. In some embodiments, the operating data displayed on the see-through display screen is dependent on a location of the wearable interface on the drilling rig. In some embodiments, the method further comprises providing two-way audio communication between the wearable frame and the drilling control module.

### BRIEF DESCRIPTION OF THE DRAWINGS

For a more detailed description of the embodiments of the present disclosure, reference will now be made to the accompanying drawings, wherein:

FIG. 1 is a schematic representation of a drilling information system including a wearable interface.

FIG. 2 is a partial schematic view of a drilling rig.

FIG. 3 is one embodiment of a wearable interface.

FIG. 4 is a partial schematic representation of a drilling information system including a wearable interface.

### DETAILED DESCRIPTION

It is to be understood that the following disclosure describes several exemplary embodiments for implementing different features, structures, or functions of the invention. Exemplary embodiments of components, arrangements, and configurations are described below to simplify the present disclosure; however, these exemplary embodiments are provided merely as examples and are not intended to limit the scope of the invention. Additionally, the present disclosure may repeat reference numerals and/or letters in the various exemplary embodiments and across the Figures provided herein. This repetition is for the purpose of simplicity and clarity and does not in itself dictate a relationship between the various exemplary embodiments and/or configurations discussed in the various figures. Moreover, the formation of a first feature over or on a second feature in the description that follows may include embodiments in which the first and second features are formed in direct contact, and may also include embodiments in which additional features may be formed interposing the first and second features, such that the first and second features may not be in direct contact. Finally, the exemplary embodiments presented below may be combined in any combination of ways, i.e., any element from one exemplary embodiment may be used in any other exemplary embodiment, without departing from the scope of the disclosure.

Additionally, certain terms are used throughout the following description and claims to refer to particular components. As one skilled in the art will appreciate, various entities may refer to the same component by different names, and as such, the naming convention for the elements described herein is not intended to limit the scope of the invention, unless otherwise specifically defined herein. Further, the naming convention used herein is not intended to distinguish between components that differ in name but not function. Additionally, in the following discussion and in the claims, the terms “including” and “comprising” are used in an open-ended fashion, and thus should be interpreted to mean “including, but not limited to.” All numerical values in this disclosure may be exact or approximate values unless otherwise specifically stated. Accordingly, various embodiments of the disclosure may deviate from the numbers, values, and ranges disclosed herein without departing from the intended scope. Furthermore, as it is used in the claims or specification, the term “or” is intended to encompass both exclusive and inclusive cases, i.e., “A or B” is intended to be synonymous with “at least one of A and B,” unless otherwise expressly specified herein.

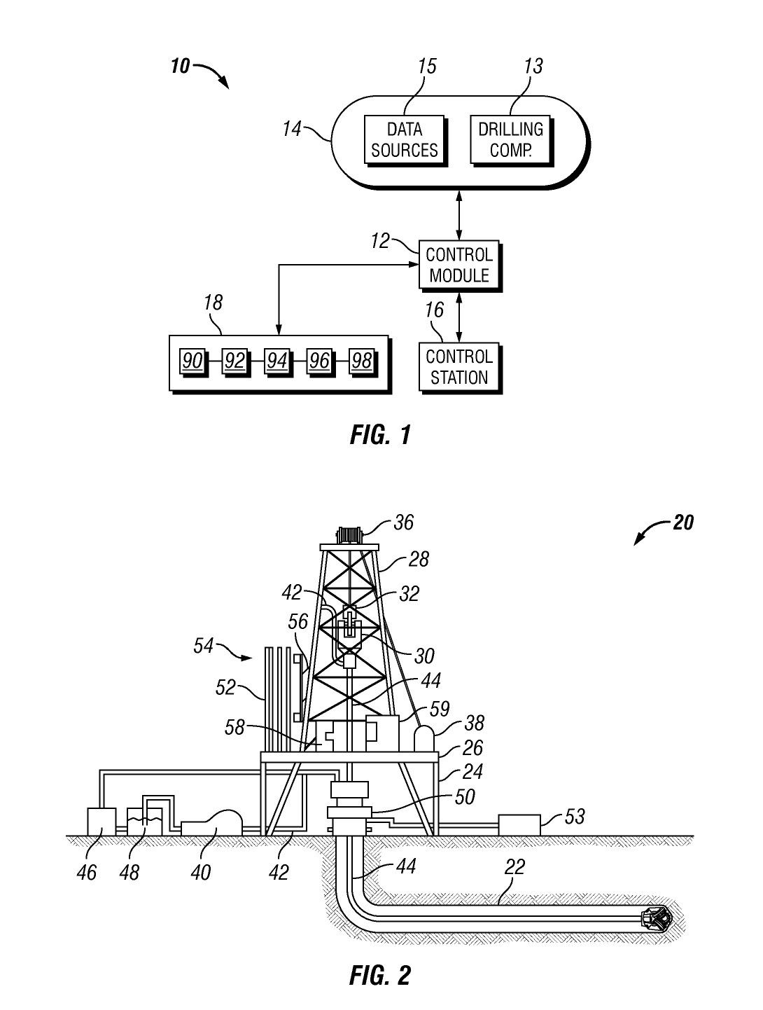

Referring initially to FIG. 1, a drilling information system 10 includes a drilling control module 12, an equipment interface 14, a control station 16, and a wearable interface 18. The equipment interface 14 is communicatively coupled to the drilling control module 12 so as to provide data from various drilling components 13 and other data sources 15 including, but not limited to, downhole sensors, drilling equipment sensors, external databases, and other data sources. The data provided by the equipment interface 14 can include information including, but not limited to, operating status, temperature, pressure, depth, rotating speed, weight, position, and other information.

Data from the equipment interface 14 is provided to the drilling control module 12 which may be operable to store, analyze, and distribute the data as needed to the control station 16 and the wearable interface 18, both of which are communicatively coupled to the drilling control module 12. In certain embodiments, drilling control module 12 is operable to process the data and issue operating instructions direct to the equipment interface 14 in an autonomous manner Control station 16 may be any type of control station, either on the drilling rig or remotely located, that allows rig personnel to observe and/or control the drilling process by inputting drilling instructions into the control station 16. In certain embodiments, the control station 16 is operable to transmit any drilling instructions back to the drilling control module 12 where those instructions are distributed via the equipment interface 14.

The wearable interface 18 may provide rig personnel access selected data, such as an operating status of certain drilling components 13 from the control module 12. For example, wearable interface 18 may be a device worn by rig personnel that selectively presents information to the wearer based on that user's location on the rig. In certain embodiments, the wearable interface 18 may be configured to present information that is relevant to the user's job duties and position on the drilling rig, thus reducing the amount of data that has to be analyzed by the user. In operation, the wearable interface 18 transmits location and identification data to the control module 12. In response, the control module 12 transmits equipment data and other relevant information back to the wearable interface 18 based on the location of the wearable interface 18 and the data selected to be provided to that particular wearable interface 18.

The wearable interface 18 includes a location sensor 90, a field-of-view sensor 92, a transmitter 94, a receiver 96, and a see-through display screen 98, each of which are coupled to a wearable frame (not shown) that can be worn by a user. The location sensor 90 generates location data indicating the location of the wearable interface 18 on the drilling rig. The field-of-view sensor 92 generates field-of-view data, which may indicate the field-of-view of the user and an identification of any drilling component located within the field-of-view of the user wearing the wearable interface 18. The transmitter 94 is operable to transmit signals, such as location data and field-of-view data, to the drilling control module 12. The receiver 96 is operable to receive signals, such as operating data and other information, from the drilling control module 12. The see-through display screen 98 is operable to display information, such as the operating data, to the user.

Referring now to FIG. 2, a drilling rig 20 may be used in the construction of a wellbore 22. The drilling rig 20 includes a sub-structure 24 that supports a drill floor 26 and a derrick 28. A top drive 30 is mounted within the derrick 28. The top drive 30 is coupled to a traveling block 32 that is moved vertically by a cable 34 that runs from the traveling block 32, through a crown block 36, and to a drawworks 38. The top drive 30 is also coupled to a mud pump 40 via a fluid supply hose 42.

The mud pump 40 pumps drilling fluid through the top drive 30 and into a drill string 44 that extends into the wellbore 22 and may be rotated by the top drive 30. Drilling fluid exits the bottom of the drill string 44 and returns to the drilling rig 20. The flow of fluid returning to the drilling rig 20 from the wellbore 22 is controlled by a blowout preventer (BOP) stack 50 and a choke manifold 53. Once the drilling fluid passes through the BOP stack 50 it is treated by solids control equipment 46 before being returned to a fluid supply 48 to be circulated back through the wellbore 22 by the mud pump 40.

As the drill string 44 is extended into and removed from the wellbore 22 sections of drill pipe 52 are stored in a pipe racking area 54 in the derrick 28. A pipe handling system 56 moves drill pipe 52 between the pipe racking area 54 and the drill floor 26. An iron roughneck 58 moves along the drill floor 26 to facilitate making and breaking connections within the drill string 44 so as to add or removed sections of drill pipe 52 from the drill string 44. The components of the drilling rig 20 may be configured to communicate with a central control system (not shown) through wired or wireless communication. In certain embodiments, some components may be fitted with radio frequency identification (RFID) tags that identify the component to the central control system or to other systems on the drilling rig 20. For example, the top drive 30 and iron roughneck 58 may have RFID tags that can be tracked to determine the position of each component on the drill floor 26.

The various systems and overall operation of the drilling rig 20 can be overseen and controlled from a driller's cabin 59 that is located on or adjacent to the drill floor 26. Rig personnel may be stationed in the driller's cabin 59 on the drill floor 26 or at operating stations at or near other components, such as the mud pump 40, the solids control equipment 46, the BOP stack 50, and the choke manifold 53.

Rig personnel may be issued a wearable interface 18 that is configured for their particular job duties and or work environment. For example, personnel on the drill floor 26 may have a wearable interface 18 that provides them with critical operating information related to the top drive 30, the pipe handling system 56, and the iron roughneck 58, such as pressure within or rotational speed of the drill string 44 or indicators of movement of the equipment on the drill floor 26. Rig personnel in other locations would have a wearable interface 18 that is configured to display information that is relevant to the equipment and area in which they work.

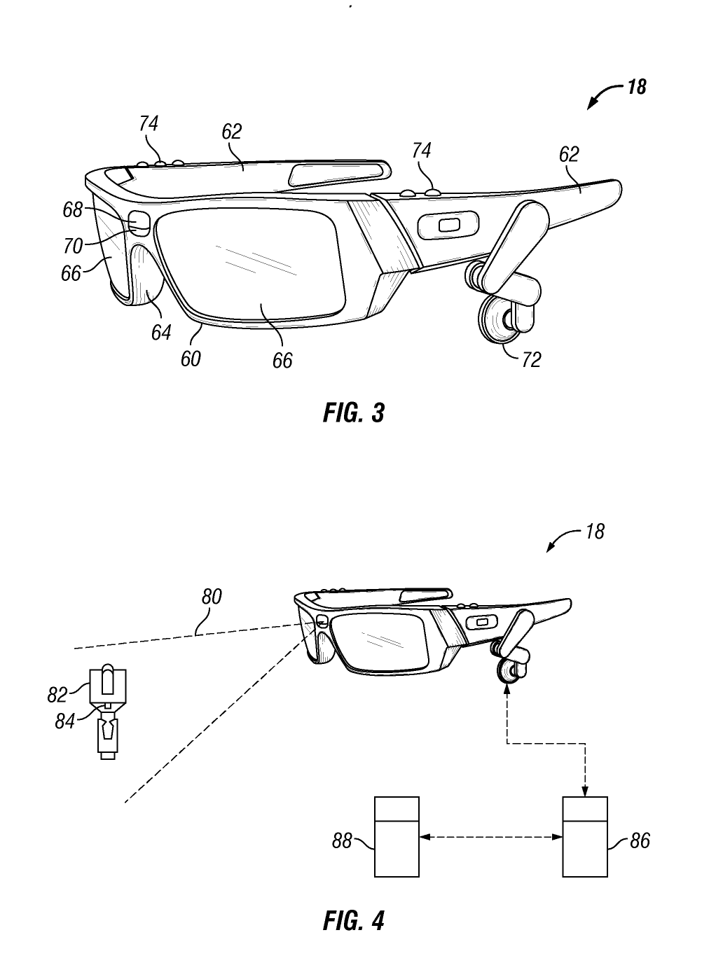

In certain embodiments, wearable interface 18 may be eye wear as shown in FIG. 3. The wearable interface 18 may include a frame 60 and temple bars 62. The frame 60 may include an integral nose piece 64 and lenses 66. The lenses 66 function as see-through display screens that may be two way reflective lenses with integral display functionality. Lenses 66 may also meet industry or governmental standards as safety glasses. The frame 60 may also include one or more cameras 68 or RFID antennas 70 that serve as location sensors and field-of-view sensors to provide data that can be used to determine the position and orientation of the wearable interface 18 relative to other equipment. The temple bars 62 may each include an ear piece 72 and one or more control buttons 74. The ear piece 72 may include a speaker for broadcasting audio to the wearer or may act as hearing protection in accordance with industry or governmental standards. The control buttons 74 may include buttons for power, brightness, alarm acknowledgement, and volume control. A microphone, wireless communication transceiver, radio transceiver, battery, RFID tag, and other electronics may also be integrated into the frame 60 or temple bars 62.

Referring now to FIG. 4, a system including a wearable interface 18 includes an interface server 86 that is connected to a control module 88. The interface server 86 includes a database of all of the wearable interfaces 18 including the data that should be provided to each wearable interface 18 based on its location on the drilling rig and rig equipment being observed. The interface server 86 then queries the control module 88 to acquire the necessary data and provides that data to each wearable interface 18 as necessary.

For example, a wearable interface 18 may be oriented so that its field of view 80 includes a top drive 82. An RFID tag 84 on the top drive transmits a signal to the RFID antenna 70 on the wearable interface 18. The wearable interface 18 transmits data identifying the wearable interface 18 and the top drive 82 (or whatever equipment is within the field of view 80) to an interface server 86. The interface server 86 then requests data from a control module 88 based on the identification of the wearable interface 18 and the top drive 82. The data is then transmitted from the interface server 86 for display by the wearable interface 18.

The interface server 86 may also be configured to continually track each wearable interface 18, via an RFID tag reading system or other means, to track the location of rig personnel. The wearable interface 18 may also serve to facilitate voice communication using radio or other wireless communication in either a station-to-station mode or as a general broadcast to all personnel, such as in the case of an emergency alarm.

While the disclosure is susceptible to various modifications and alternative forms, specific embodiments thereof are shown by way of example in the drawings and description. It should be understood, however, that the drawings and detailed description thereto are not intended to limit the disclosure to the particular form disclosed, but on the contrary, the intention is to cover all modifications, equivalents and alternatives falling within the spirit and scope of the present disclosure.

What is claimed is:

1. A drilling information system comprising:
a drilling component having an operating status;
a drilling control module communicatively coupled to the drilling component and operable to store data indicating the operating status of the drilling component; and
a wearable interface communicatively coupled to the drilling control module, wherein the wearable interface is operable to selectively indicate the operating status of the drilling component.

2. The drilling information system of claim 1, wherein the wearable interface comprises:
a wearable frame;
a location sensor coupled to the wearable frame, wherein the location sensor generates location data indicating a location of the wearable frame on a drilling rig;
a field-of-view sensor coupled to the wearable frame, wherein the field-of-view sensor generates field-of-view data indicating a field-of-view of a user wearing the wearable frame;
a transmitter mounted to the wearable frame and operable to transmit the location data and field-of-view data to the drilling control module;
a receiver mounted to the wearable frame and operable to receive operating data from the drilling control module, wherein the operating data indicates the operating status of the drilling component that is disposed within the field-of-view; and
a see-through display screen mounted to the wearable frame and operable to display the operating data proximate to the drilling component.

3. The drilling information system of claim 2, further comprising an RFID antenna coupled to the wearable frame, wherein the RFID antenna is operable to detect an RFID tag mounted on the drilling component.

4. The drilling information system of claim 2, further comprising an RFID tag coupled to the wearable frame and operable to identify the wearable interface.

5. The drilling information system of claim 2, further comprising a speaker coupled to the wearable frame.

6. The drilling information system of claim 5, further comprising a microphone coupled to the wearable frame.

7. The drilling information system of claim 2, wherein the operating data displayed on the see-through display screen is dependent on a location of the wearable interface on the drilling rig.

8. The drilling information system of claim 1, wherein the wearable interface provides hearing and eye protection to a user.

9. A wearable interface comprising:
a wearable frame;
a location sensor coupled to the wearable frame, wherein the location sensor generates location data indicating a location of the wearable frame on a drilling rig;
a field-of-view sensor coupled to the wearable frame, wherein the field-of-view sensor generates field-of-view data indicating a field-of-view of a user wearing the wearable frame;
a transmitter mounted to the wearable frame and operable to transmit the location data and field-of-view data to a drilling control module;
a receiver mounted to the frame and operable to receive operating data from the drilling control module, wherein the operating data indicates an operational status of a drilling component that is disposed within the field-of-view; and
a see-through display screen mounted to the wearable frame and operable to display the operating data proximate to the drilling component.

10. The wearable interface of claim 9, further comprising an RFID antenna coupled to the wearable frame, wherein the RFID antenna is operable to detect an RFID tag mounted on the drilling component.

11. The wearable interface of claim 9, further comprising an RFID tag coupled to the wearable frame and operable to identify the wearable interface.

12. The wearable interface of claim 9, further comprising a speaker coupled to the wearable frame.

13. The wearable interface of claim 12, further comprising a microphone coupled to the wearable frame.

14. The wearable interface of claim 9, wherein the operating data displayed on the see-through display screen is dependent on a location of the wearable interface on the drilling rig.

15. The wearable interface of claim 9, wherein the wearable interface provides hearing and eye protection to a user.

16. A method for displaying data comprising:
disposing a wearable frame on a user located on a drilling rig;
generating location data indicating a location of the user on the drilling rig;
generating field-of-view data indicating a field-of-view of the user;
transmitting the location data and field-of-view data to a drilling control module;
receiving operating data from the drilling control module, wherein the operating data indicates an operational status of a drilling component that is disposed within the field-of-view; and
displaying the operating data on a see-through display screen coupled to the wearable frame.

17. The method of claim 16, further comprising receiving data identifying the drilling component from an RFID tag mounted on the drilling component.

18. The method of claim 16, further comprising transmitting data identifying the wearable frame with an RFID tag mounted on the wearable frame.

19. The method of claim 16, wherein the operating data displayed on the see-through display screen is dependent on a location of the wearable interface on the drilling rig.

20. The method of claim 16, further comprising providing two-way audio communication between the wearable frame and the drilling control module.
